(19) United States
(12) Patent Application Publication
(10) Pub. No.: US 2002/0004664 A1
(43) Pub. Date: Jan. 10, 2002

(54) Ear candle enhancer

(76) Inventor: Laura Ann Gerstein, Boca Raton, FL (US)

Correspondence Address: Robert M. Downey, ROBERT M. DOWNEY, P.A., Suite 350, 150 E. Palmetto Park Road, Boca Raton, FL, 33432, US

(21) Appl. No.: 09/767,356

(22) Filed: Jan. 23, 2001

Related U.S. Application Data
(60) Provisional application No. 60/216,537, filed on Jul. 6, 2000.

(51) Int. Cl. A61F 9/00

(52) U.S. Cl. 606/162

(57) ABSTRACT

A device for enhancing the efficiency and safety of using ear candles to remove wax and other debris from the ear canal includes a tubular body having a proximal end zone and an opposite distal end zone. The distal end zone is fitted with a holder for releasable attachment to the bottom end of an ear candle so that the end of the ear candle is maintained in sealed, free flowing communication with a passageway extending through the tubular body. The proximal end zone includes a flexible and pliable tubular extension which is specifically adapted for comfortable, sealing engagement with the inner ear to provide an air tight seal between the inner ear and the passageway of the device, and thereby maintaining a vacuum within the device as the ear candle burns, resulting in removal of wax and other debris from the ear canal and through the passageway of the device and into the ear candle.

### BACKGROUND OF THE INVENTION

1. Field of the Invention

The present invention is directed to a device for use with an ear candle to clean the ear canal of a user and, more particularly, to an ear candle holding device which extends between the end of an ear candle and the user's ear canal while maintaining a sealed passage therebetween.

2. Background of the Invention

Ear candles have been used for thousands of years to remove excessive ear wax, debris, and other undesirable substances from the ear canal. It is believed that this method of cleaning the ear canal, commonly referred to as “ear candling,” originated with the ancient Egyptians who used hollow reeds. The modern day candle is made of muslin cloth strips, dipped in wax, and coiled into a long slender cone having a length of approximately 12 inches.

Use of ear candles to remove excessive ear wax and other debris from the ear canal helps to maintain and/or improve the quality of a person's hearing. Over time, the effects of allergies and sicknesses (colds, flu, sinus problems, etc.) can cause a build up of wax and other debris in the ear canal. Also, extensive use of various appliances, including telephones and hearing appliances (hearing aids), can induce build up of wax in the ear canal. Eventually, the build up of wax and other debris can be so great as to cause a natural “ear plug” that can dramatically alter a person's hearing capabilities. In many instances, an effective cleaning of the ear canal using ear candles can lead to a much larger range of hearing frequency, thereby eliminating the need for a hearing aid. Ear candles are also used to enhance drainage of toxins that may be attempting to escape the body through the ear canal. For instance, the Eustachian tube connects the middle ear with the nose and throat. Should the Eustachian tube become clogged with excessive ear wax, an infection may develop. Infection can also occur when one of the body's systems is trying to purge toxins from inside the body, in liquid form, through the ear canal.

In use, the top end of the ear candle is lit, and as it burns, smoke begins to billow out of the bottom of the cone-shaped ear candle. The bottom end is then placed snugly into the ear canal to form an air tight seal between the open bottom end of the ear candle and the ear canal. As the ear candle continues to burn, air is forced back up through the hollow, cone-shaped ear candle, creating a warm air current through the ear candle towards the top burning end. This results in a warm vacuum, creating suction at the bottom end of the ear candle and within the ear canal. The warm air melts the ear wax and the created suction pulls the wax and other debris from within the ear canal and through the bottom end of the ear candle. The liquid wax hits the sides of the cooler candle, the wax solidifies on the sides of the inside of the candle. As the ear candle continues to burn, some of the ear wax is burned off while the remainder collects within the hollow candle, particularly near the bottom end.

Ear candling has proven to be far more effective than other conventional methods of cleaning the ears. For instance, repeated water cleansings tend to create a prolonged moist environment that can invite growth of bacteria and fungus which may lead to an infection of the middle or inner ear. Also, because wax is a natural repellant of water, cleaning the ear canal with water is not entirely effective in removing ear wax and other debris. Moreover, water inevitably becomes trapped in the ears after cleansing, causing an annoying noise and minor discomfort.

While ear candling is a highly effective method for cleaning the ear canal, there are some problems which are encountered when using ear candles. Specifically, as the ear candle burns down and becomes shorter, the flame on the end of the candle burns close to the ear and hair of the user. This can certainly create a concern for both the comfort and safety of the user. For this reason, many users of ear candles tend to extinguish them early, leaving a significant portion of the ear candle unused. This in turn requires use of a greater number of ear candles to accomplish a complete ear cleaning. Another problem associated with ear candles is the difficulty in obtaining a good seal between the candle and the ear canal in order to maintain a vacuum. Also, as wax begins to melt within the candle, the hot wax can sometimes return to the user's ear canal causing severe discomfort and possible injury.

The present invention is specifically designed to overcome the problems encountered while using ear candles to clean the ear canal. Moreover, the present invention is designed to increase the overall effectiveness and efficiency of ear candling, while providing for greater comfort and safety throughout the ear cleaning process. In particular, the device of the present invention provides the following benefits:

1. Creates a stronger and more sustained vacuum between the ear candle and ear canal for greater efficiency and effectiveness. This results in greater amounts of ear wax and other debris being removed per candle.

2. Allows the user to burn more of each ear candle, thereby reducing the number of ear candles needed to complete an ear cleaning procedure, and thus lowering the overall cost of the ear cleaning procedure.

3. Provides an increased length between the ear canal and the ear candle, thereby allowing for hot ear wax to solidify on return from the ear candle towards the ear canal and reducing the risk of hot ear wax returning into the ear.

4. Provides protection from the ash and flame of the candle by positioning the candle further away from the user's ear and hair.

5. Provides for greater comfort and a more efficient seal with the inside of the user's ear, thereby resulting in greater tolerance when multiple ear candles are needed to complete an ear cleaning procedure.

6. Extra length provided by the device position the ear candle further from the ear, hair and head of the user, thereby making it easier to use ear candles without the assistance of others.

7. Extra length provided by the device also positions the flame further away from the user's ear, thereby reducing the intensity of the heat that reaches the ear cavity and making it possible to use more ear candles in succession during an ear cleaning procedure.

### BRIEF DESCRIPTION OF THE DRAWINGS

For a fuller understanding of the nature of the present invention, reference should be made to the following detailed description taken in conjunction with the accompanying drawings in which:

FIG. 1 is a side view of the device of the present invention;

FIG. 2 is a side view of the device shown attached to an ear candle;

FIG. 3 is a partial sectional view taken along line 3-3 of FIG. 2; and

FIG. 4 is an elevational view showing the device in use and extending between an ear candle and the ear canal of a user.

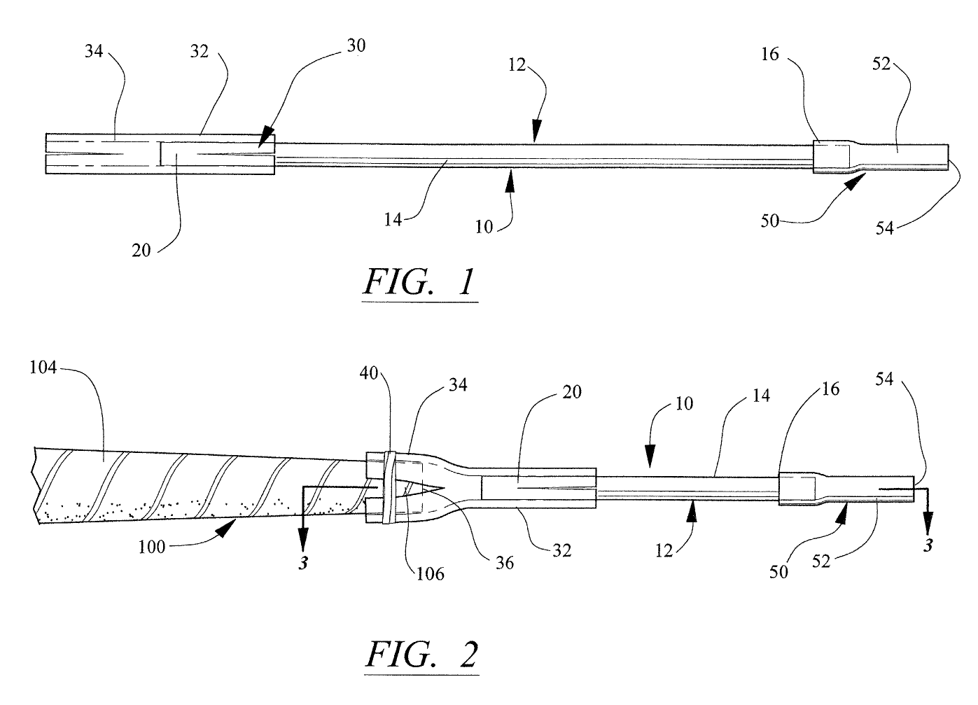

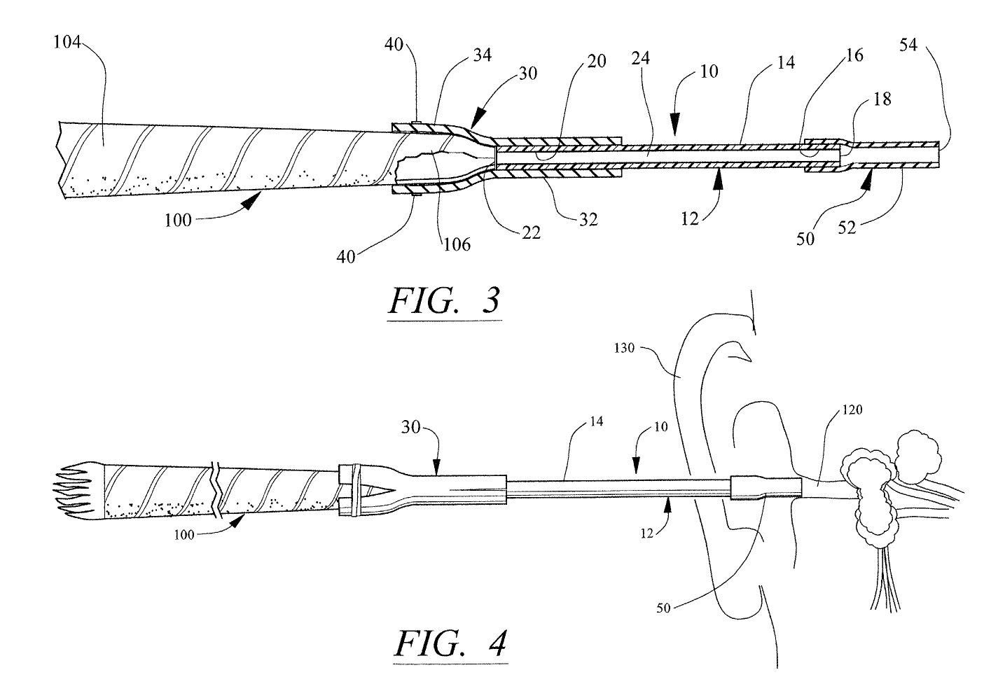

Like reference numerals refer to like parts throughout the several views of the drawings.

### DETAILED DESCRIPTION OF THE PREFERRED EMBODIMENT

Referring to the several views of the drawings, the ear candle enhancer device is shown and is generally indicated as 10. The device 10 is specifically intended for use with an ear candle 100 in order to clean the ear canal 120 of a user's ear 130. The ear candle is typically made of muslin cloth strips which are dipped into wax (e.g., beeswax) and coiled into a long slender cone of approximately 12 inches in length, with a top open end 104 and a bottom open end 106. Typically, the top open end 104 has a circumference of approximately one-half inch to three quarters of an inch, while the bottom open end 106 is of smaller circumference so that the hollow ear candle tapers slightly from the top end 104 to the bottom end 106.

The device 10 includes a base member 12 defined by a hollow tubular body 14 having a proximal end zone 16 terminating at a first open end 18 and a distal end zone 20 terminating at a second open end 22. A passageway 24 extends through the hollow tubular body between the opposite first and second open ends. In a preferred embodiment, the tubular body 14 is provided with a length ranging between 1 inch to 20 inches and can be cut to any desired size. The circumference of the tubular body is preferably in the range of between ⅛inch to 2 inches.

Holding means 30 are provided on the distal end zone 20 of the base member for releasably holding the bottom end zone of the ear candle therein so that the bottom open end of the ear candle is disposed in free flowing communication with the second open end 22 of the tubular body and the passageway 24 extending therethrough. In a preferred embodiment, the holding means 30 includes a segment of tubing 32 fitted over the distal end zone 20 of the base member 12 and snug about an outer surface of the distal end zone so as to maintain a sealed engagement therewith. A portion 34 of the tubular segment 32 extends beyond the second open end 22 of the tubular body and is split longitudinally on opposite sides, as at 36, in order to accommodate receipt of various size ends of ear candles therein. Once the bottom end 106 of the ear candle is received and nested within the extending portion 34 of the tubular segment 32, as shown in FIG. 2, a rubber band 40 is placed about the split extending portion 34 of the tubular segment in a manner which holds the end of the candle within the extending portion of the tubular segment.

It is preferable that the tubular segment 32 be of a greater flexibility than the tubular body 14 of the base member 12. The tubular segment fitted to the distal end zone is preferably in the range of between ⅛inch to 12 inches in length and can be cut to any size within that range, as desired by the user. Further, in a preferred embodiment, the circumference of the tubular segment is between ⅛inch to ¾inch and length ranges between ⅛inch and 12 inches. It should be noted that while the circumference of the base tubular body 14 may be greater than the circumference of the tubular segment 32 in a relaxed state, the flexible elastomeric material of the tubular segment 32 permits stretching so that the tubular segment can be fitted over the distal end zone 20 of the tubular body of the base member in snug engagement therewith, as described above.

The proximal end zone 16 of the tubular body 14 is fitted with seal means 50 for sealing engagement with the user's ear canal 120. In a preferred embodiment, the seal means comprises a segment of flexible tubing 52 which is fitted over the proximal end zone 16 of the tubular body 14 in snug fitting relation thereto. In a preferred embodiment, the flexible tubing 52 has a length ranging between ⅛inch to 12 inches and can be cut to any size within that range, as desired by the user. The flexible tubing segment 52 further includes a circumference ranging between ⅛inch to ¾inch. In a preferred embodiment, the flexible tubing segment 52 is of greater flexibility and elasticity than the tubular segment 32 on the opposite distal end zone. The segment of flexible tubing 52, defining the seal means 50, is specifically intended to be inserted within the user's ear 130 for sealing engagement with the ear canal 120. The soft flexible rubber material of the tubing segment 52 is particularly ideal for maintaining an air tight seal with the ear canal to thereby maintain vacuum within the device during the entire time that the ear candle is burning, thereby improving the efficiency of removing ear wax and other debris from the ear canal. And, unlike inserting the end of an ear candle within the ear which is still and can scratch the inner ear canal when attempting to make an air tight fit, the soft pliable material of the seal means on the device provides a more comfortable and safer engagement with the inner ear.

In use, the ear candle 100 is first attached to the device 10 by inserting the bottom end 106 of the ear candle within the tubular segment 32 of the holding means 30. To do this, the rubber band 40 is first moved away from the extending portion 34 and towards the tubular body 14. This can be done by simply rolling the rubber band 40 downwardly along the tubular segment 32. Next, the split extending portion 34 is separated to permit gentle insertion of the bottom end 106 of the ear candle within the tubular segment 32 until the bottom end 106 is snug therein. Next, the rubber band 40 is gently rolled upwardly along the tubular segment 32 until the rubber band is wrapped around the split extending portion 34 and the bottom end 106 of the ear candle received therein. This secures the ear candle to the device 10. It should be noted that attachment of the ear candle to the device should be done gently and with care, as ear candles are delicate and can collapse, which would restrict the vacuum flow through the bottom open end 106 and through the candle.

Once the ear candle is fitted to the device 10, the top end 104 of the ear candle is lighted, preferably using a cigarette lighter. Soon after the ear candle is lit, white smoke begins to flow out of the open end 54 of the tubing segment 52. When this happens, the segment of tubing 52 is placed within the ear and positioned until the smoke stops escaping from the ear. When properly positioned in the ear, the smoke is forced to rise up through the passageway of the device and through the ear candle, thus creating an air current or vacuum flow which is needed to clean the ear. At this point, the user should hear a crackling noise in the ear caused by the warm air current melting the ear wax. If the user does not hear the crackling noise, it may mean that there is an ineffective seal between the seal means 50 and the ear canal 120. The user should continue to manipulate the device until the seal means 50, and particularly the open end 54 of the segment of tubing 52, creates an air tight seal within the inner ear canal. Otherwise, a lack of a crackling noise may indicate that there is a clog either in the device 10 or within the ear canal 120. This will need to be cleared before proceeding any further with the ear candling procedure.

As the ear candle continues to burn down creating an ash, the ash may fall off of the candle. Thus, it is preferable to use a drop cloth or towel to protect the area and clothing of the user. When the ear candle has burned down to about 2 inches from the bottom end 106, the ear candle should be extinguished by dunking the burning flame into a container of water. Once extinguished, the remaining portion of the ear candle can be removed from the device 10 and placed on a paper plate or napkin for disposal.

At this stage, another ear candle should be attached to the device 10 and lighted as quickly as possible so that melted and loosened wax in the ear canal does not have time to harden. It is important to note that the first candle begins to loosen the wax or debris within the ear canal, and the remaining candles gain efficiency with each successive one, to effectively remove all wax and debris from the ear canal. Typically, four to six ear candles are needed to complete an ear cleaning procedure for one ear. It is advisable to wait at least 48 hours before repeating treatment in the same ear. Also, it is advisable to wait at least 24 hours before treating the other ear in the event that the user experiences a temporary reduction in hearing immediately following the cleaning procedure. Typically, if there is a reduction in the hearing, it lasts for no more than one day.

While the instant invention has been shown and described in accordance with a practical and preferred embodiment thereof, it is recognized that departures from the instant disclosure are contemplated within the spirit of the invention and, therefore, the scope of the invention should not be limited except as defined within the following claims as interpreted under the doctrine of equivalents.

What is claimed is:

1. A device for use with an ear candle to clean the ear canal of a user, said device comprising:
a base member including a tubular body having a proximal end zone terminating at a first open end and a distal end zone terminating at a second open end opposite to said first open end and a passageway extending through said base tube between said opposite open ends;
holding means on said distal end zone for releasably holding the ear candle on the device so that an end of the ear candle is maintained in sealed, free flowing communication with said second open end and said passageway of said tubular body;
seal means on said proximal end zone for sealing engagement with the user's ear and said seal means being structured and disposed for maintaining sealed communication between the ear canal and the end of the ear candle during use of the device; and
whereby burning the ear candle creates a vacuum within the device to cause removal of substances from the ear canal and through the passageway.

2. The device as recited in claim 1 wherein said holding means includes a flexible tubular segment fitted to said distal end of said base member.

3. The device as recited in claim 2 wherein said flexible tubular segment includes adjustment means for accommodating a range of sizes of ear candle ends therein.

4. The device as recited in claim 3 wherein said adjustment means includes one or more longitudinal splits in said flexible tubular segment.

5. The device as recited in claim 4 wherein said holding means further comprises an elastomeric band fitted about said flexible tubular segment and movable there along to permit receipt and removal of said end of said ear candle within said flexible tubular segment, said elastomeric band being further structured and disposed to apply pressure about said flexible tubular segment to thereby hold the end of the ear candle within said flexible tubular segment.

6. The device as recited in claim 1 wherein said seal means includes a flexible and pliable tubular member fitted to said proximal end zone of said base member.

7. A device for use with an ear candle to clean the ear canal of a user, said device comprising:
a base member including a tubular body having a proximal end zone terminating at a first open end and a distal end zone terminating at a second open end opposite to said first open end and a passageway extending through said base tube between said opposite open ends;
holding means on said distal end zone for releasably holding the ear candle on the device so that an end of the ear candle is maintained in sealed, free flowing communication with said second open end and said passageway of said tubular body, said holding means including a flexible tubular segment including a portion extending from said second open end of said tubular body, said extending portion being structured and disposed for removable receipt of the end of the ear candle therein;
said holding means further including securing means for maintaining said end of said ear candle within said extending portion of said flexible tubular segment, said securing means including an elastomeric band fitted about said flexible tubular segment and being structured and disposed for applying a grasping force about said extending portion to thereby hold the end of the ear candle therein;
seal means on said proximal end zone for sealing engagement with the user's ear and said seal means being structured and disposed for maintaining sealed communication between the ear canal and the end of the ear candle during use of the device, said seal means including a flexible and pliable tubular member fitted to and extending from said proximal end zone of said tubular body; and
whereby burning the ear candle creates a vacuum within the device to cause removal of substances from the ear canal and through the passageway.
